(19) United States
(12) Patent Application Publication
(10) Pub. No.: US 2014/0242673 A1
(43) Pub. Date: Aug. 28, 2014

(54) CHEMICALLY DEFINED CULTURE MEDIUM FOR FERMENTATION TO PRODUCE SUCCINIC ACID AND APPLICATION THEREOF

(71) Applicant: NANJING UNIVERSITY OF TECHNOLOGY, Nanjing, Jiangsu (CN)

(72) Inventors: Min Jiang, Nanjing (CN); Yongian Xi, Nanjing (CN); Jiuhua Zhang, Nanjing (CN); Rong Xu, Nanjing (CN); Wenyu Dai, Nanjing (CN); Kequan Chen, Nanjing (CN); Ping Wei, Nanjing (CN)

(21) Appl. No.: 14/352,320

(22) PCT Filed: Sep. 14, 2012
(86) PCT No.: PCT/CN2012/081415

§ 371 Date: Apr. 18, 2014

(30) Foreign Application Priority Data
Nov. 11, 2011 (CN) 201110356173.3

(51) Int. Cl. C12N 1/20

(52) U.S. Cl. CPC C12N 1/20
USPC 435/252.8; 435/253.6

(57) ABSTRACT

This invention belongs to the technical field of industrial microorganism fermentation and relates to a type of chemical defined medium used for succinic acid production by fermentation and its application. The chemical defined medium of this invention used for succinic acid production by fermentation includes conventional compositions and critical growth factor compositions. Said critical growth factor compositions include biotin or 5-ALA, niacin, amino acid, and methionine. This formula determines four critical growth factors of succinic acid fermentation through reasonable technical means. The culture medium is free of complicated and expensive nutritious compositions such as yeast powder and peptone. Reliance on expensive vitamin by production of succinic acid through fermentation in defined medium is avoided, so that production cost of succinic acid can be greatly lowered, and subsequent separation process can be simplified, constituting a key step of industrial production of succinic acid using chemical defined medium.

### FIELD OF THE INVENTION

This invention belongs to the technical field of industrial microorganism fermentation and relates to a type of chemical defined medium used for succinic acid production by fermentation and its application, in particular, succinic acid production by fermentation using chemical defined medium, determination of minimum consumption of biotin in the culture medium, and replacement of biotin by 5-aminolevulinate (5-ALA) for succinic acid production.

### BACKGROUND OF THE INVENTION

Defined medium used for microorganism fermentation is prepared by sequential addition of accurately weighed high purity chemical reagents in distilled water, so that the compositions (including microelements) and their quantities are clearly known. Defined medium is normally used for research in laboratory that has relatively high requirements on quantities, e.g. nutrition, metabolism, heredity, identification, and bioassay etc.

During production of succinic acid by fermentation of microorganism that generates succinic acid in defined medium, as main component of organic nitrogen source, biotin has the function of utmost importance in growth and metabolism of microorganism that generates succinic acid. Since chemical defined medium has definite compositions and is free of complicated and expensive nutritious compositions such as yeast powder and peptone, it favors investigation of wild type strains that produce succinic acid for which metabolic pathway is not clearly known. Besides, since chemical defined medium consists of some inorganic salt ions, a few amino acids, and a few vitamins, its cost is lower than that of ordinary composite culture medium. For production of succinic acid by fermentation using chemical defined medium, most costs of later separation can be saved. However, up to now, there has been no report of research in production of succinic acid at lowered cost of chemical defined medium, and the cost of chemical defined medium used for production of succinic acid by fermentation is still high.

In existing technology, for strains Actinobacillus succinogenes 130Z and Mannheimia succiniciproducens MBEL55E that produce succinic acid, chemical defined medium has been screened out. However, these chemical synthetic culture media in existing technology have complicated compositions, and contain various amino acids, vitamins, and metallic salts (liquid). Therefore, existing technology fails to provide technical guide for production of succinic acid by industrial fermentation using chemical defined medium.

### SUMMARY OF THIS INVENTION

One technical purpose of this invention is to determine and provide a compounding formula of chemical defined medium used for production of succinic acid by fermentation. This culture medium compounding formula can optimize consumption of the most expensive vitamin: biotin, to reach the minimum value of this consumption required to maintain normal fermentation of succinic acid, and reduce waste. According to growth characteristics and metabolic pathway characteristics of strains that produce succinic acid, a substituting substance for biotin has been finally found, namely 5-ALA, used for fermentation. Reliance on expensive vitamin by production of succinic acid through fermentation in defined medium is avoided, so that production cost of succinic acid can be greatly lowered, and subsequent separation process can be simplified, constituting a key step of industrial production of succinic acid using chemical defined medium.

Another technical purpose of this invention is to provide a method of production of succinic acid by fermentation of microorganism that produces succinic acid using this chemical defined medium.

To realize these technical purposes, the technical scheme of this invention is as follows.

I. A type of chemical defined medium used for production of succinic acid by fermentation, wherein its conventional compositions include 10˜90 g/L of separately sterilized carbon source, 1˜3 g/L of disodium fumarate, 2˜4 g/L of KH₂PO₄, 0.2˜0.4 g/L of MgCl₂.6H₂O, 0.2˜0.4 g/L of CaCl₂, and 1˜2 g/L of NaCl; wherein it also includes the following critical growth factor compositions: 10˜20 mg/L of biotin or 0.1˜1 mg/L of 5-ALA, 25˜40 mg/L of niacin, 0.87˜1.2 mg/L of amino acid, and 0.11˜0.22 mg/L of methionine; wherein sterilization is performed at pH value of 7.0 and 121° C. for 15 min.

Further, said carbon source includes (but not limited to) glucose, arabinose, cane sugar, or mixed carbohydrate obtained from pre-treated stalk.

Further, said chemical defined medium also includes conventional composition of neutralizing agent, which may be (but not limited to) sodium hydroxide, sodium bicarbonate, or ammonia.

II. A method of production of succinic acid by fermentation using the chemical defined medium of this invention, including steps of activation of microorganism germ seed, cultivation of seed, and cultivation in fermenter.

Further, said step of germ seed activation is: plate streaking of germ seed in slant culture medium, followed by activation cultivation at 37° C. in anaerobic incubator for 24 h.

Said step of seed cultivation is: transfer activated germ seed to seed culture medium, for cultivation at 37° C. for 1012 h, to be used as seed liquid later.

Said step of cultivation in fermenter is: transfer activated seed liquid and sterilized glucose into fermenter; inoculum size shall be 5˜10% (volume ratio); stirring speed shall be 200 rpm; allow fermentation at 37° C.; amount of CO₂ connected shall be 0.25 vvm.

Succinic acid producing strain described by this invention includes any microorganism germ seed that produces succinic acid by anaerobic fermentation, e.g. NJ113 (Actinobacillus succinogenes NJ113), Escherichia coli, corynebacterium glutamicum, and Mannheimia succiniciproducens MBEL55E.

Beneficial effects of this invention are:

This invention determines and provides a compounding formula of chemical defined medium used for succinic acid production by fermentation. This formula determines four critical growth factors of succinic acid fermentation through reasonable technical means. The culture medium is free of complicated and expensive nutritious compositions such as yeast powder and peptone. This formula also optimizes consumption of the most expensive vitamin: biotin, to reach the minimum value of this consumption required to maintain normal fermentation of succinic acid, reducing waste. According to growth characteristics and metabolic pathway characteristics of strains that produce succinic acid, a substituting substance for biotin has been finally found, namely 5-ALA, used for fermentation. Reliance on expensive vitamin by production of succinic acid through fermentation in defined medium is avoided, so that production cost of succinic acid can be greatly lowered, and subsequent separation process can be simplified, constituting a key step of industrial production of succinic acid using chemical defined medium.

### DETAILED DESCRIPTION OF PARTICULAR EMBODIMENTS OF THE INVENTION

The following preferred embodiments provide detailed description of this invention, but do not limit application of this invention.

### Preferred Embodiment 1

To investigate the effect of critical factors on succinic acid production described by this invention can use any microorganism germ seed used for production of succinic acid using anaerobic fermentation in existing technology. This preferred embodiment uses NJ113 (Actinobacillus succinogenes NJ113), which has been patented (authorized publication No.: CN100537744C).

First, significance and effect of various amino acids, vitamins, and metallic microelements on bacterial growth and metabolism has been investigated on defined medium. Particular operation and cultivation conditions are as follows:

\textcircled{1} Preparation of amino acid mixed liquids: Remove each one type of amino acid from the 18 types of amino acid being tested, to prepare a mixed liquid of remaining 17 types of amino acid of suitable concentration (suitable concentration refers to the concentration at which the substance of lowest solubility is completely dissolved), as well as a mixed liquid containing all 18 types of amino acid. Quantity of each such liquid is 100 mL. After filtration sterilization by 0.22 μm sterile filtering head, keep these liquids for later use. Refer to Attached Table 1 for contents of various amino acids in the culture medium.

\textcircled{2} Preparation of vitamin mixed liquids: Using the method in step 0, remove each one type of vitamin from the 10 types of vitamin being tested, to prepare a mixed liquid of remaining 9 types of vitamin of suitable concentration (suitable concentration refers to the concentration at which the substance of lowest solubility is completely dissolved), as well as a mixed liquid containing all 10 types of vitamin. Quantity of each such liquid is 100 mL. After filtration sterilization by 0.22 μm sterile filtering head, keep these liquids for later use. Refer to Attached Table 1 for contents of various vitamins in the culture medium.

\textcircled{3} Preparation of metallic microelement mixed liquids: Using the method in step \textcircled{1}, remove each one type of metal salt from the 12 types of metal salt being tested, to prepare a mixed liquid of remaining 11 types of metal salt of suitable concentration (suitable concentration refers to the concentration at which the substance of lowest solubility is completely dissolved), as well as a mixed liquid containing all 12 types of metal salt. Quantity of each such liquid is 100 mL. After sterilization at 121° C. for 15 min, keep these liquids for later use. Refer to Attached Table 1 for contents of various metal salts in the culture medium.

Fermentation conditions of each stage:

Germ seed activation: plate streaking of germ seed in slant culture medium, followed by activation cultivation at 37° C. in anaerobic incubator for 24 h.

Seed cultivation: transfer activated germ seed to seed culture medium, for cultivation at 37° C. for 10˜12 h, to be used as seed liquid later.

Cultivation in fermenter: transfer activated seed liquid and sterilized glucose into fermenter; inoculum size shall be 10% (volume ratio); stirring speed shall be 200 rpm; allow fermentation at 37° C.; amount of CO₂ connected shall be 0.25 vvm.

Slant culture medium (g/L): 10 of glucose (separately sterilized), 5 of yeast extract, 10 of NaHCO₃, 9.6 of NaH₂PO₄.2H₂O, 15.5 of K₂HPO₄.3H₂O, and 20 of agar; sterilized at pH of 7.0 and 121° C. for 15 min.

Seed culture medium (g/L): 10 of glucose (separately sterilized), 5 of yeast extract, 10 of NaHCO₃, 9.6 of NaH₂PO₄.2H₂O, and 15.5 of K₂HPO₄.3H₂O; sterilized at pH of 7.0 and 121° C. for 15 min.

Conventional compositions of fermentation culture medium (g/L): 90 of glucose (separately sterilized), 1 of disodium fumarate, 2 of KH₂PO₄, 0.2 of MgCl₂.6H₂O, 0.2 of CaCl₂, and 1 of NaCl; sterilized at pH of 7.0 and 121° C. for 15 min; sodium carbonate adopted as neutralizing agent.

In a 100 mL serum bottle, add 22 mL of fermentation culture medium and 1 mL of Actinobacillus succinogenes NJ113 (CGMCC 1716) seed liquid that has been washed by sterilized water; sterilize at 121° C. for 15 min; and then add 4 mL of reserved mixed liquid not containing a type of amino acid, 0.5 mL of reserved mixed liquid containing all vitamins being tested, 1 mL of reserved mixed liquid containing all types of metal salt being tested, and 1.5 mL of sterilized 600 g/L glucose, so that after inoculation, the total volume reaches 20 mL and glucose concentration is 30 g/L.

Use the same method to prepare fermentation culture medium that lacks a certain type of vitamin or metal salt.

Carry out fermentation of above culture media under the following conditions:

At mechanical shaker speed of 200 rpm and 37° C., allow fermentation for 30 h and then detect content of succinic acid in the fermentation liquid. It has been found that when the culture medium lacks glutamic acid, biotin, niacin, or methionine, bacterial growth is relatively poor, large amount of glucose remains, and succinic acid production is very low. Results are given in Table 1.

TABLE 1
Effect of fermentation with missing growth factor

| Missing growth | | | |
|---|---|---|---|
| factor | OD₆₆₀ | Residual glucose, g/L | Succinic acid, g/L |
| Glutamic acid | 0.4 | 8.1 | 0.5 |
| Biotin | 0.3 | 6.9 | 0.7 |
| Niacin | 0.2 | 5.6 | 0.1 |
| Methionine | 0.6 | 4.3 | 1.3 |

After critical growth factors are determined, their effect on production of succinic acid by fermentation has been investigated, using the following method:

Prepare 100 mL of mixed liquid containing aforesaid four critical factors, so that their concentrations in the culture medium are as those given in Attached Table 1. Filter and sterilize this liquid for later use. In a 3 L fermenter, add 1600 mL of water, 15 g/L of Na₂HPO₄, and 4 g/L of KH₂PO₄. Sterilize at 121° C. for 15 min and then add 200 mL of 500 g/L glucose, 100 mL of Actinobacillus succinogenes NJ113 (CGMCC 1716) seed liquid after being washed by sterilized water, and 100 mL of reserved mixed liquid of growth factors.

Carry out fermentation experiment of above culture medium and conventional succinic acid production culture medium under the same fermentation conditions:

After sterilization, add 5% Actinobacillus succinogenes NJ113 (CGMCC 1716) seed liquid for stirring at 300 rpm and fermentation at 37° C. Amount of CO₂ connected is 0.25 vvm. Use Na₂CO₃ to control fermentation liquid pH value to 6.8. After 48 h of fermentation, measure succinic acid concentration. Refer to Table 2.

TABLE 2
Results of succinic acid production by fermentation
using different culture media

| | | Glucose | Succinic |
|---|---|---|---|
| | Nutritious substance | consumption | acid (g/L) |
| Conventional culture | 10 g/L yeast powder | 50 | 36.8 |
| medium | | | |
| Culture medium of this | Four critical growth | 50 | 45.0 |
| preferred embodiment | factors described | | |
| | above | | |

### Preferred Embodiment 2

This preferred embodiment reduces biotin consumption in the culture medium. Particular method of change of fermentation culture medium is described below.

Germ seed: Escherichia coli (strain from patent of publication No. CN102154339A)

Cultivation in fermenter: transfer activated seed liquid and sterilized glucose into fermenter; inoculum size shall be 5% (volume ratio); stirring speed shall be 200 rpm; allow fermentation at 37° C.; amount of CO₂ connected shall be 0.25 vvm.

Conventional compositions of fermentation culture medium (g/L): 30 of glucose (separately sterilized), 2 of disodium fumarate, 3 of KH₂PO₄, 0.3 of MgCl₂.6H₂O, 0.23 of CaCl₂, and 1 of NaCl; sterilized at pH of 7.0 and 121° C. for 15 min; sodium carbonate adopted as neutralizing agent.

In a 100 mL serum bottle, add 15 g/L of Na₂HPO₄, 4 g/L of KH₂PO₄, 13.1 mL of water, and 1 mL of Actinobacillus succinogenes NJ113 (CGMCC 1716) seed liquid after being washed by sterilized water; sterilize at 121° C. for 15 min, and then add 4 mL of reserved mixed liquid containing corresponding concentrations of amino acids, 0.5 mL of mixed liquid of vitamins including niacin and biotin so that final biotin concentration in the fermentation shake-flask is 0 mg/L, 2 mg/L, 4 mg/L, 6 mg/L, and 8 mg/L respectively, 1 mL of reserved mixed liquid containing all metal salts being tested, and 0.4 mL of sterilized 500 g/L glucose solution, so that total volume after inoculation reaches 20 mL and glucose concentration is 10 g/L.

Fermentation experiment of above culture media containing different concentrations of biotin has been carried out under the same fermentation conditions as preferred embodiment 1:

Under different biotin concentrations, different fermentation results are given in Table 3:

TABLE 3
Results of production of succinic acid by fermentation
at different biotin concentrations

| Biotin concentration | | Residual | Succinic |
|---|---|---|---|
| (mg/L) | OD₆₆₀ (g/L) | glucose (g/L) | acid (g/L) |
| 0 | 0.8 | 22.5 | 0 |
| 2 | 2.28 | 0 | 18.8 |
| 4 | 2.31 | 0 | 18.6 |
| 6 | 2.18 | 0 | 17.7 |
| 8 | 2.19 | 0 | 19.4 |
| 10 | 2.25 | 0 | 18.7 |
| 20 | 2.13 | 0 | 18.5 |

According to identical germ seed, fermentation conditions, and experiment method of this preferred embodiment, biotin consumption in the culture medium is further lowered, to concentrations of 50 μg/L, 100 μg/L, 150 μg/L, 200 μg/L, and 300 μg/L respectively, for fermentation experiment:

Under further lowered biotin concentrations in Defined Medium, results of fermentation are given in Table 4:

TABLE 4
Results of production of succinic acid by fermentation
under different biotin concentrations

| Biotin concentration | | Residual | Succinic |
|---|---|---|---|
| (μg/L) | OD₆₆₀ (g/L) | glucose (g/L) | acid (g/L) |
| 50 | 0.97 | 23 | 1.6 |
| 100 | 2.78 | 2.2 | 15.8 |
| 150 | 2.89 | 2.6 | 16.2 |
| 200 | 2.89 | 2.5 | 16.8 |
| 300 | 2.90 | 2.8 | 16.7 |

### Preferred Embodiment 3

In this preferred embodiment, biotin (10˜20 mg/L) in culture medium is replaced by 0.1˜1 mg/L of 5-ALA, for fermentation experiment.

The microorganism adopts large Mannheimia succiniciproducens MBEL55E (strain from patents of publication No. EP2096177 and No. EP2199304).

Cultivation in fermenter: transfer activated seed liquid and sterilized glucose into fermenter; inoculum size shall be 7% (volume ratio); stirring speed shall be 200 rpm; allow fermentation at 37° C.; amount of CO₂ connected shall be 0.25 vvm.

Conventional compositions of fermentation culture medium (g/L): 10 of mixed carbohydrate obtained from pre-treated stalk (measured total carbohydrate concentration), 3 of disodium fumarate, 4 of KH₂PO₄, 0.4 of MgCl₂.6H₂O, 0.4 of CaCl₂, and 2 of NaCl; sterilize at pH value of 7.0 and 121° C. for 15 min; ammonia is adopted as neutralizing agent.

Results of fermentation using chemical defined medium in which biotin is completed replaced by 5-ALA are given in Table 5.

Data prove that use of cheap 5-ALA can realize the highest output of succinic acid that can be obtained using biotin.

TABLE 5
Results of production of succinic acid by fermentation
with biotin replaced by 5-ALA in synthetic cultivation

| 5-ALA concentration | | Residual | Succinic |
|---|---|---|---|
| (mg/L) | OD660 (g/L) | glucose (g/L) | acid (g/L) |
| 0.1~1 | 2.48 ± 0.22 | 3.4 | 15.6 ± 0.2 |

### Preferred Embodiment 4

Basically same as above preferred embodiment, the difference being: said microorganism adopts corynebacterium glutamicum (strain from patent of publication No. CN101984046A) and said carbon source is arabinose.

### Preferred Embodiment 5

Compounding formula of the chemical defined medium of this invention is finally determined.

Fermentation culture medium: 10˜90 g/L of separately sterilized glucose, 1˜3 g/L of disodium fumarate, 2˜4 g/L of KH₂PO₄, 0.2˜0.4 g/L of MgCl₂.6H₂O, 0.2˜0.4 g/L of CaCl₂, 1˜2 g/L of NaCl, 10˜20 mg/L of biotin or 0.1˜1 mg/L of 5-ALA, 25˜40 mg/L of niacin, 0.87˜1.2 mg/L of amino acid, and 0.11˜0.22 mg/L of methionine; sterilize at pH value of 7.0 and 121° C. for 15 min.

ATTACHED TABLE 1

| | Concentration | Final concentration |
|---|---|---|
| | in preferred | in fermentation |
| Composition | embodiment 1 (mg/L) | culture medium (mg/L) |
| D-biotin | 10 | 10~20 |
| Niacin | 25 | 25~40 |
| Folic acid | 10 | 10~30 |
| D-pantothenic acid | 25 | 25~50 |
| Benadon | 50 | 50~70 |
| Vitamin B12 | 0.5 | 0.5~1 |
| Vitamin B1 | 25 | 25~50 |
| P-benzaminic acid | 25 | 25~50 |
| Flavine | 25 | 25~50 |
| Lipoic acid | 25 | 25~50 |
| L-alanine | 0.43 | 0.43~0.73 |
| L-arginine | 0.22 | 0.22~0.50 |
| L-cysteine | 0.20 | 0.20~0.50 |
| L-glutamic acid | 0.87 | 0.87~1.20 |
| L-glycine | 0.15 | 0.15~0.30 |
| L-histidine | 0.08 | 0.08~0.16 |
| L-isoleucine | 0.28 | 0.28~0.50 |
| L-leucine | 0.53 | 0.53~0.75 |
| L-methionine | 0.11 | 0.11~0.22 |
| L-serine | 0.24 | 0.24~0.48 |
| L-threonine | 0.22 | 0.22~0.48 |
| L-valine | 0.31 | 0.31~0.60 |
| L-tyrosine | 0.11 | 0.11~0.25 |
| L-phenylalanine | 0.28 | 0.28~0.50 |
| L-tryptophan | 0.09 | 0.09~0.20 |
| L-lysine | 0.21 | 0.21~0.50 |
| L-proline | 0.11 | 0.11~0.25 |
| Aminotriacetic acid | 1500 | 1500~2000 |
| Bitter salt | 3000 | 3000~3500 |
| Sulfuric acid | 500 | 500~1000 |
| monohydrate | | |
| Ferrous sulfate | 100 | 100~150 |
| heptahydrate | | |
| Calcium chloride | 100 | 100~150 |
| dihydrate | | |
| Zinc chloride | 13 | 13~20 |
| Blue copperas | 10 | 10~20 |
| Potassium/aluminium | 10 | 10~20 |
| sulfate dodecahydrate | | |
| Boric acid | 10 | 10~20 |
| Sodium molybdate | 25 | 25~40 |
| Nickel chloride | 25 | 25~40 |
| hexahydrate | | |
| Sodium tungstate | 25 | 25~40 |
| dihydrate | | |
| Sodium selenate | 10 | 10~20 |

1-8. (canceled)

9. A medium composition used for succinic acid production through fermentation, comprising 1090 g/L of carbon source, 13 g/L of disodium fumarate, 24 g/L of KH2PO4, 0.20.4 g/L of MgCl2.6H2O, 0.20.4 g/L of CaCl2, 12 g/L of NaCl, 1020 mg/L of biotin or 0.11 mg/L of 5-ALA, 2540 mg/L of niacin, 0.871.2 mg/L of amino acid, and 0.110.22 mg/L of methionine, said medium composition being sterilized at pH 7.0 and 121 for 15 min.

10. The medium composition according to claim 9, wherein said carbon source is selected from the group consisting of glucose, arabinose, cane sugar, and mixed carbohydrate obtained from pre-treated stalk.

11. The medium composition according to claim 9, further comprising sodium hydroxide, sodium bicarbonate, or ammonia.

12. A method of producing the medium composition according to claim 9, comprising steps of (a) activation of microorganism germ seed, (b) cultivation of seed, and (c) cultivation in fermenter.

13. The method according to claim 12, wherein said step of activation of microorganism germ seed is via plate streaking of germ seed in slant culture medium followed by cultivation at 37 in an anaerobic incubator for 24 hours.

14. The method according to claim 12, wherein said step of cultivation of seed is conducted by transferring said activated germ seed to a seed culture medium for cultivation at 37 for 10˜12 hours to obtain an activated seed liquid.

15. The method according to claim 12, wherein said step of cultivation in fermenter is conduced by transferring said activated seed liquid and sterilized glucose into a fermenter with an inoculum ratio of 5˜10% by volume and cultivating under stirring at 200 rpm, 37, and CO₂ 0.25 vvm.

16. The method according to claim 12, wherein said microorganism germ seed is a species selected from the group consisting of Actinobacillus succinogenes NJ 113, Escherichia coli, corynebacterium glutamicum, and Mannheimia succiniciproducens MBEL55E.
